(19) United States
(12) Patent Application Publication
(10) Pub. No.: US 2008/0194880 A1
(43) Pub. Date: Aug. 14, 2008

(54) Process For the Preparation of Polymorphs of Mesotrione

(76) Inventors: Stephen Dawson, Huddersfield (GB); Neil George, West Yorkshire (GB); Ian Kevin Jones, West Yorkshire (GB); Julie Marie Wichert, Bucks, AL (US)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 11/574,205

(22) PCT Filed: Aug. 3, 2005
(86) PCT No.: PCT/GB0505/003069

§ 371 Date: Oct. 16, 2007

(30) Foreign Application Priority Data
Aug. 26, 2004 (GB) 0419075.7

(51) Int. Cl. C07C 317/14

(52) U.S. Cl. 568/31

(57) ABSTRACT

A process for selectively controlling the crystallisation of thermodynamically stable Form (1) or kinetically stable Form (2) polymorphs of mesotrione from an aqueous mesotrione solution, said method comprising adjusting the pH of the mesotrione solutions to a value wherein said thermodynamically stable Form (1) or kinetically stable Form (2) mesotrione is ultimately obtained is disclosed.

The present invention relates to a process for selectively controlling the crystallisation of polymorphic forms of mesotrione. The invention further relates to the use of pH to control the polymorphic formation. The invention further relates to a process for converting one polymorphic form to another. The invention still further relates to one particular polymorphic form of mesotrione.

The protection of crops from weeds and other vegetation that inhibits crop growth is a constantly recurring problem in agriculture. To help combat this problem, researchers in the field of synthetic chemistry have produced an extensive variety of chemicals and chemical formulations effective in the control of such unwanted growth. Chemical herbicides of many types have been disclosed in the literature and a large number are in commercial use. Commercial herbicides and some that are still in development are described in The Pesticide Manual, 13^{th} Edition, published 2003 by the British Crop Protection Council.

Many herbicides also damage crop plants. The control of weeds in a growing crop therefore required the use of so-called ‘selective’ herbicides, which are chosen to kill the weeds while leaving the crop undamaged. In practice, few herbicides are fully selective, in that they will kill all the weeds and leave the crop untouched at a particular application rate. The use of most selective herbicides is actually a balance between applying enough herbicides to acceptably control most of the weeds and causing only minimal crop damage. One known selective herbicide is mesotrione (2-(4-methylsulphonyl-2-nitrobenzoyl)cyclohexane-1,3-dione).

It is known that some organic compounds occur in only one crystal structure, while others occur in two or more crystal structures (known as polymorphs). It is not possible to predict the number of different polymorphs a given compound will have, nor the physical, chemical and biological properties thereof.

Crystallisation of mesotrione is carried out by a pH shift in a predominantly aqueous solution whereby the soluble salt is converted to the insoluble free acid resulting in high yield. It has recently been discovered that mesotrione exists in two polymorphic forms: the thermodynamically stable form, known as Form 1; and the metastable form, known as Form 2.

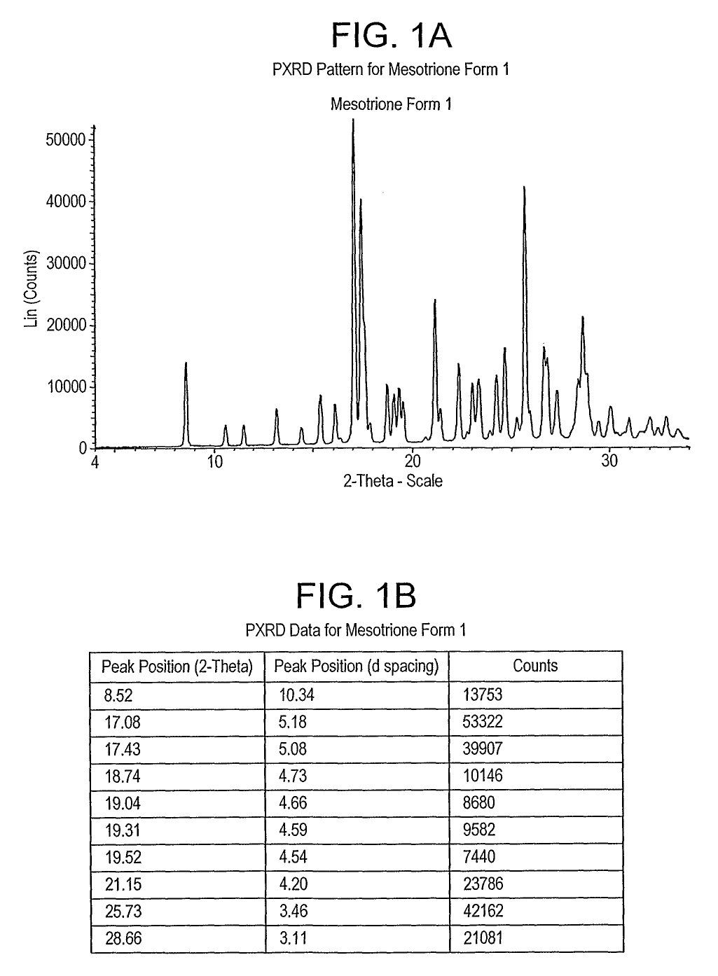

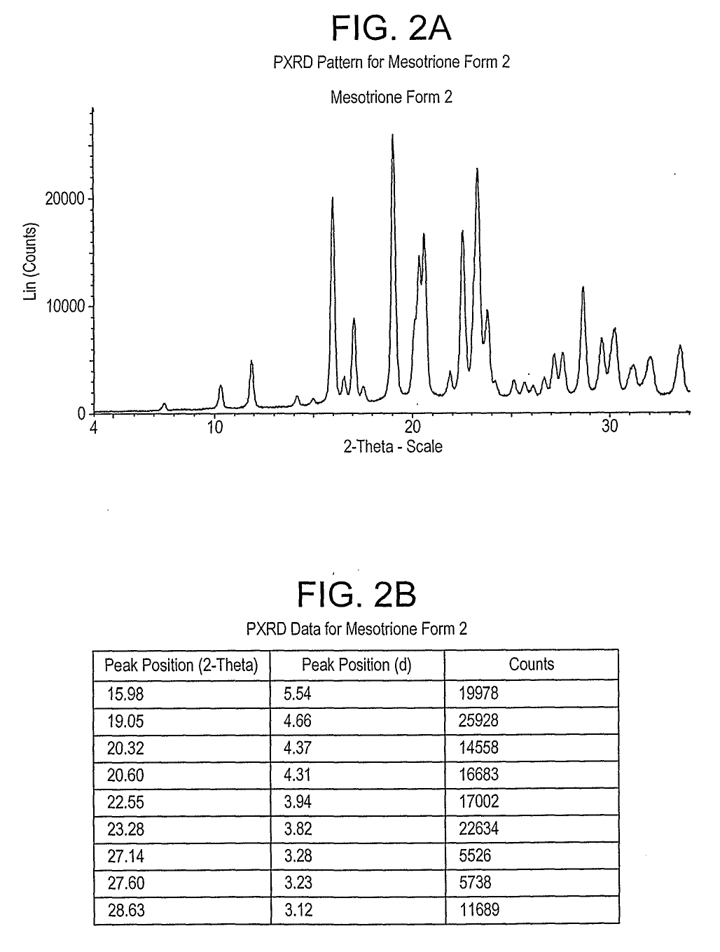

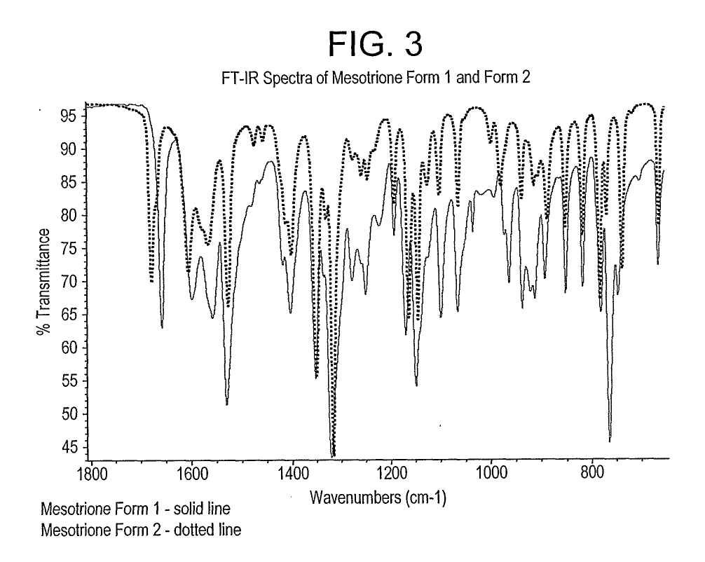

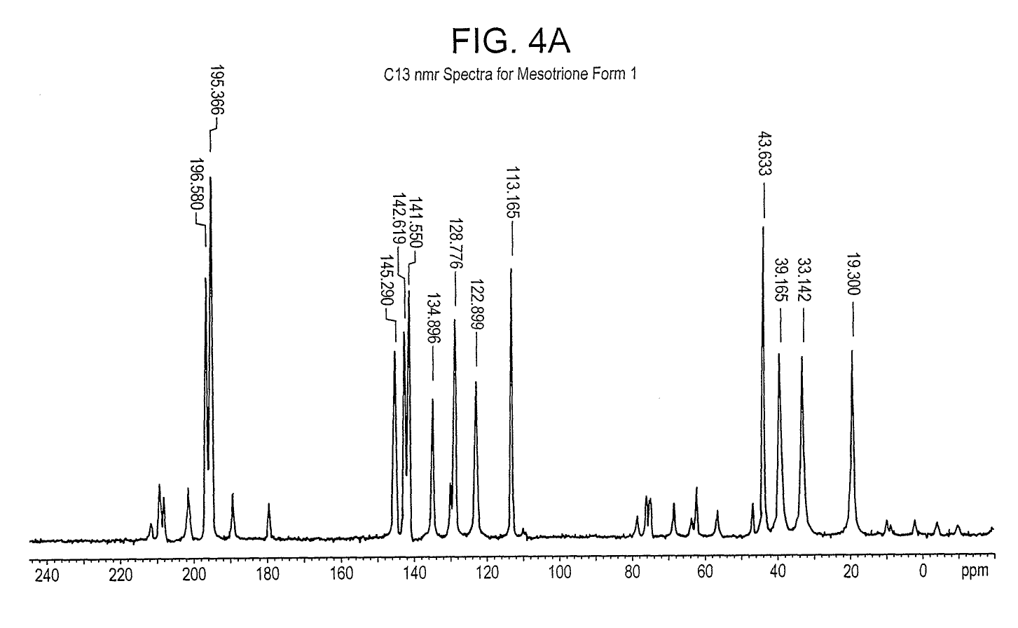

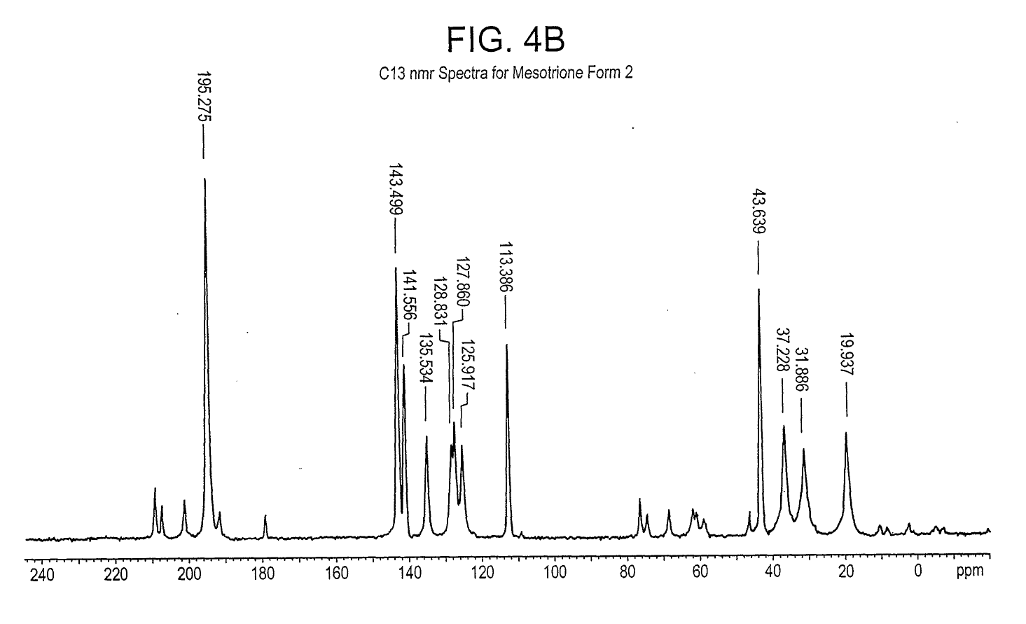

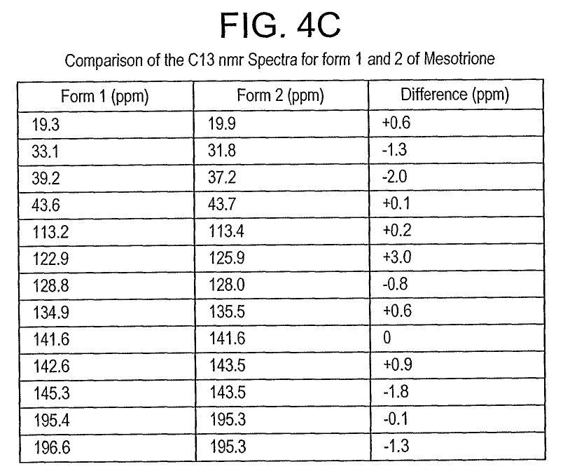

For aqueous crystallisation a large difference in size between Form 1 and Form 2 was seen and this is a very useful technique for assessing the presence of Form 2. The powder XRD patterns and data for the two polymorphic forms are also distinctly different and are shown in FIGS. 1 and 2. FIG. 3 compares the infra-red patterns of the two polymorphic forms clearly showing distinctive differences in the pattern and hence crystal structure. Furthermore, the two polymorphs give significant differences in their solid state ¹³C nmr measurements, FIGS. 4A, 4B and 4C.

Form 1 is the polymorphic form currently used in commercially available formulations. However, due to the size of the crystals, milling is required to reduce the crystal size when formulating into an agrochemically acceptable composition. Form 2 is already of a size that would be suitable for formulating into an agrochemically acceptable composition. However, Form 2 is thermodynamically unstable and would gradually convert to Form 1; consequently any formulation prepared therefrom would be unstable and would aggregate and settle.

A further problem exists in that Form 1 is currently the form used in preparing agrochemically acceptable formulation, but during the manufacturing process, Form 2 is readily made when mesotrione is recrystallised in aqueous solution. Due to Form 2 being very fine, it is difficult to filter and production time is lost while trying to remove it from the system. If the Form 2 material obtained during recrystallisation cannot be converted to Form 1, then it must be disposed of, resulting in lost revenue and inefficient production processes.

Therefore, a first object of the invention is to provide a process for selectively controlling which polymorph is obtained and is stable.

A second object of the invention is to provide a process for readily converting Form 2 polymorph into Form 1 polymorph.

Accordingly, the present invention provides a process for selectively controlling the crystallisation of thermodynamically stable Form 1 or kinetically stable Form 2 polymorphs of mesotrione from an aqueous mesotrione solution, said method comprising adjusting the pH of the mesotrione solution to a value wherein said thermodynamically stable Form 1 or kinetically stable Form 2 mesotrione is ultimately obtained.

Suitably, the pH of the mesotrione solution is first increased to a pH of ≧7, suitably ≧10, and preferably ≧12. The pH can be increased by the addition of a suitable base, for example NaOH, pyridine, triethylamine, Mg(OH)₂, NH₄OH etc. The addition of the base results in a salt of mesotrione being formed which has a high solubility, ensuring that mesotrione is fully solubilised and that no mesotrione remains out of solution.

In one embodiment of the invention, the pH is adjusted to ≦3.0, resulting in the thermodynamically stable Form 1 mesotrione being obtained. Suitably, the pH is adjusted to pH ≦2.5, and preferably to pH 2±0.5.

In a second embodiment of the invention, the pH is adjusted to greater than 3.0 resulting in the kinetically stable Form 2 mesotrione being obtained. Suitably, the pH is adjusted to a value between about greater than 3.0 and about 5.5, preferably between 3.5 and 5.5. The upper value of pH is dependent on which particular mesotrione salt is in solution.

The adjustment in pH is suitably carried out by the addition of acid to the mesotrione solution. Suitably, the acid is selected from the group consisting of HCl, H₂SO₄, HNO₃ etc; preferably HCl.

In some cases it may be beneficial to add some Form 1 seed crystals to the solution after reducing the pH in order to assist crystallisation of Form 1.

In certain cases, for example at a pH of 3.0 or slightly below, an increase in temperature can and crystallisation of Form 1. Furthermore, the presence of salt and/or solvent can and crystallisation of Form 1.

Suitably, the process is carried out at a temperature of ≧25° C., preferably ≧40° C.

A second aspect of the invention provides a process for converting Form 2 mesotrione to Form 1 mesotrione, said process comprising reducing the pH of a Form 2 mesotrione suspension to a pH of ≦1.0. Suitably, the pH is adjusted to pH ≦2.5, and preferably to pH 2±0.5.

In one embodiment of this aspect of the invention, the Form 2 mesotrione has previously been isolated and is resuspended in an appropriate solvent, for example water.

In a second embodiment of this aspect of the invention, the Form 2 mesotrione has been formed as a result of the manufacturing process, and has not been isolated; it is therefore already suspended in the mother liquor.

Suitably, the pH of the Form 2 mesotrione suspension is first increased to a pH of ≧7, suitably ≧10, and preferably ≧12. The pH can be increased by the addition of a suitable base, for example NaOH, etc. The addition of the base results in a salt of mesotrione being formed, which has a high solubility, resulting in the Form 2 mesotrione going into solution.

The reduction in pH is suitably carried out by the addition of acid to the mesotrione suspension. Suitably, the acid is selected from the group consisting of HCl, H₂SO₄, HNO₃ etc; preferably HCl.

In some cases it may be beneficial to add some Form 1 seed crystals to the Form 2 mesotrione suspension after reducing the pH in order to assist crystallisation of Form 1.

In certain cases, for example at a pH of 3.0 or slightly below, an increase in temperature can aid crystallisation of Form 1. Furthermore, the presence of salt and/or solvent can aid crystallisation of Form 1.

Suitably, the process is carried out at a temperature of ≧25° C., preferably ≧40° C.

As discussed hereinbefore, the invention arises from the realisation that pH can be used to control the formation of one particular polymorph over the other or to convert one polymorph to the other. Accordingly, a further aspect of the invention provides the use of pH to control the crystallisation of polymorphs of mesotrione.

The presence of Form 2 mesotrione has not until now been disclosed. Accordingly, a further aspect of the invention provides a polymorph of mesotrione, wherein said polymorph is characterised by a powder X Ray diffraction pattern and data as given in FIG. 2 and C¹³ nmr data as given in FIGS. 4B and 4C.

The invention will now be described further by reference to the following examples.

### EXAMPLE 1

This is an example of the conversion of already isolated Form 2 mesotrione to Form 1 mesotrione in the presence of solvent. A 10% solution of Form 2 in water was made at different pH values ranging from 2 to 6. If seeded, a seed concentration of 2% Form 1 relative to the Form 2 mesotrione concentration was used. A 1:5 ratio of xylene to mesotrione was used in these experiments. Samples were analyzed for polymorph form after the time shown in table. Actual conversion time may be less than shown. The results are shown in Table 1.

TABLE 1

| | | | | | Dominant |
|---|---|---|---|---|---|
| Example | Temp | Time | | | Polymorph |
| No. | (° C.) | (hr) | pH | Seed | Form (IR) |
| 1A | 21 | 6.3 | 2.0 | yes | 1 |
| 1B | 21 | 6.3 | 2.9 | yes | 2 |
| 1C | 21 | 6.3 | 4.1 | yes | 2 |
| 1D | 21 | 6.3 | 5.9 | yes | 2 |
| 1E | 21 | 6.3 | 2.5 | yes | 1 |
| 1F | 21 | 6.3 | 5.1 | yes | 2 |
| 1G | 21 | 23.3 | 2.0 | yes | 1 |
| 1H | 21 | 23.3 | 2.9 | yes | 2 |
| 1I | 21 | 23.5 | 5.9 | yes | 2 |
| 1J | 21 | 23.5 | 2.5 | yes | 1 |
| 1K | 21 | 23.5 | 2.0 | no | 1 |
| 1L | 21 | 22.0 | 2.0 | no | 1 |
| 1M | 21 | 22.0 | 4.0 | no | 2 |

### EXAMPLE 2

This is an example of converting in process Form 2 mesotrione to Form 1 mesotrione. Form 2 material was made in the plant via a process upset. Samples of the Form 2 slurry from the crystallizer were taken to the lab where the pH of the solution was adjusted to 2.0 and the material was heated to 40-50 C while agitated. The results are shown in Table 2.

TABLE 2

| | Example No. | | | | |
|---|---|---|---|---|---|
| | 2A | 2B | 2C | 2D | 2E |
| Temp of | 20° C. | 40° C. | 50° C. | 50° C. | 40° C. |
| conversion | | | | | |
| Hours to | Did not | 4 | 1.4 | 1.8 | 4 |
| convert | convert | | | | |
| | after 4-5 | | | | |
| | days | | | | |
| pH of | 3.2 | 2.0 | 2.0 | 2.9 | 2.0 |
| conversion | | | | | |
| Agitated | no | yes | yes | yes | yes |
| Seeded | no | no | no | no | no |

### EXAMPLE 3

This is an example of converting already isolated Form 2 mesotrione to Form 1 mesotrione by placing the Form 2 material in process filtrate, adding different amounts of TEA and NaCl, adjusting the pH to 2.0, and heating the material to 40-50 C. The results are shown in Table 3.

TABLE 3

Example No.

| | 3A | 3B | 3C | 3D |
|---|---|---|---|---|
| Temp of | 40 | 50 | 50 | 50 |
| conversion | | | | |
| Hours to | 17 | 1 | 5 | 4 |
| convert | | | | |
| pH of | 1.8 | 2.0 | 2.0 | 1.8 |
| conversion | | | | |
| Ratio | | 1:1.3 | 1:1.3 | 1:1.3 |
| meso:TEA | | | | |
| Ratio | | 2:1 | 2:1 | 2:1 |
| meso:NaCl | | | | |
| Agitated | yes | yes | yes | yes |
| Seeded | no | no | no | no |

### EXAMPLE 4

This is an example of converting already isolated Form 2 mesotrione to Form 1 mesotrione by placing the Form 2 material in process filtrate, adjusting the pH to 2.0, and heating the material to 40-50 C.

TABLE 4

| | Example No. | | | | | |
|---|---|---|---|---|---|---|
| | 4A | 4B | 4C | 4D | 4E | 4F |
| Temp of | 22 | 22 | 30 | 50 | 50 | 50 |
| conversion | | | | | | |
| Hours to convert | Did not | Did not | Did not | 2 | 2 | 2 |
| | convert | convert | convert | | | |
| | after | after 48 hrs | after | | | |
| | 52 hrs | | 18 hrs | | | |
| pH of conversion | 3.6 | 3.6 | 3.6 | 2.0 | 1.8 | 1.8 |
| Agitated | yes | yes | yes | yes | yes | no |
| Seeded | yes | yes | yes | no | no | no |

### EXAMPLE 5

### Isolation of Mesotrione from a Crude Enolate Solution

Plant mesotrione enolate suspension was filtered to remove any excess solid enolate. 50 ml of the filtered solution was placed in a reaction flask and heated to 40° C. A pH probe was placed in the vessel to monitor the pH and the pH was reduced by adding 10% hydrochloric acid in a controlled manner over 20 minutes (the addition can also be done over 5 minutes and Form 1 is still isolated) to 2.8. The crystals were allowed to stir for 20 minutes before being isolated by filtration under reduced pressure, washed with water and sucked dry on the filter. The polymorphic form of the product was confirmed by FT-IR and PXRD as Mesotrione Form 1.

### EXAMPLE 6

### Isolation of Mesotrione from a Laboratory Prepared Enolate Solution

The filtrates were placed in a reaction flask, stirred and crystals of Mesotrione added. The pH of the slurry was measured and increased to 10.5 by the addition of 48% sodium hydroxide. The slurry was stirred for 60 minutes and the excess crystals were removed by filtration.

20 ml of the enolate solution was placed in a reaction flask stirred and heated to 40° C. A pH probe was place in the solution and the pH was reduced to 2.6 by the controlled addition of 10% hydrochloric acid over 20 minutes. The resulting crystals were stirred for a further 60 minutes before being collected by filtration under reduced pressure, washed with water and sucked dry on the filter. The polymorphic form of the crystals was determined as form 1 by FT-IR and PXRD.

### EXAMPLE 7

### Polymorphic Stability of Pure Mesotrione

1.6 g of the re-crystallised mesotrione crystals were stirred with water (30 ml) in a reaction flask and the pH increased to 12 by the addition of sodium hydroxide. 1.5 ml of 10% hydrochloric acid was added over 15 minutes to reduce the pH of the solution to pH ranges between 1 and 4. The suspension was stirred and the polymorphic form of the crystals was determined as Form 2 by optical microscopy and FT-IR. Below pH 2.5, Form 2 transformed to Form 1 within 1 hour. At pH 3 Form 2 was stable but converted to Form 1 when seeded with Form 1 over four hours. At pHs between 3.5 and 4 the suspension could be heated to 40° C. and 1% by weight of Form 1 seeds added and the Form 2 crystals would not transform to Form 1. Periodically over the next 3 weeks the polymorphic form of the suspension was determined and was always found to be Form 2. After 3 weeks the monitoring ceased on a regular basis, samples taken several months later still showed that the crystals had not transformed to Form 1.

### EXAMPLE 8

### Stabilising Form 2 Mesotrione in a 0.05% Rhodasurf DA630 Solution

0.5 g of Form 2 mesotrione crystals were stirred with a 0.05% Rhodasurf DA630 (15 ml) in a reaction flask. A pH probe was placed in the system and the pH was increased to 11.5 by the addition of 0.6 ml of 10% sodium hydroxide solution. The pH of the solution was reduced to 5.5 by the addition of 0.74 g of Form 2 mesotrione. The polymorphic form of the mesotrione was monitored periodically by microscopy and FT-IR. After 10 days the mesotrione was still predominately Form 2; Form 1 had nucleated but bad not grown.

1. A process for selectively controlling the crystallisation of thermodynamically stable Form 1 or kinetically stable Form 2 polymorphs of mesotrione from an aqueous mesotrione solution, said method comprising adjusting the pH of the mesotrione solutions to a value wherein said thermodynamically stable Form 1 or kinetically stable Form 2 mesotrione is ultimately obtained.

2. A process according to claim 1, wherein the pH of the mesotrione solution is first increased to a pH of ≧7.

3. A process according to claim 1, wherein the pH is adjusted to ≦3.0, resulting in the thermodynamically stable Form 1 mesotrione being obtained.

4. A process according to claim 1, wherein the pH is adjusted to greater than 3.0, resulting in the kinetically stable Form 2 mesotrione being obtained.

5. A process according to claim 1, wherein the reduction in pH is carried out by the addition of acid to the mesotrione solution.

6. A process according to claim 1, wherein Form 1 seed crystals are added to the solution after adjusting the pH in order to assist crystallisation of Form 1.

7. A process according to claim 1 wherein said process is carried out at a temperature of ≧25° C.

8. A process for converting Form 2 mesotrione to Form 1 mesotrione, said process comprising reducing the pH of a Form 2 mesotrione suspension to a pH of ≦3.0.

9. A process according to claim 8, wherein the Form 2 mesotrione has previously been isolated and is resuspended in an appropriate solvent.

10. A process according to claim 8, wherein the Form 2 mesotrione has been formed as a result of the manufacturing process and is already suspended in the manufacturing process mother liquor.

11. A process according to claim 8, wherein the pH of the Form 2 suspension is first increased to a pH of ≧7.

12. A process according to claim 8, wherein the reduction in pH is carried out by the addition of acid to the mesotrione suspension.

13. A process according to claim 8, wherein Form 1 seed crystals are added to the Form 2 suspension after reducing the pH in order to assist crystallisation of Form 1.

14. A process according to claim 8, wherein the process is carried out at a temperature of ≧25° C.

15. (canceled)

16. A polymorph of mesotrione, wherein said polymorph is characterised by a powder X Ray diffractions pattern and data as given in FIG. 2.
